(19) United States
(12) Patent Application Publication
(10) Pub. No.: US 2024/0307360 A1
(43) Pub. Date: Sep. 19, 2024

(54) ORAL DISPERSIBLE FILM OF MITRAGYNINE

(71) Applicant: LUCKY 4U EXIMS, Tirupati (IN)

(72) Inventors: Surya Pavan Kumar AVVA, Tirupati (IN); Raghavi KARUMURI, Hyderabad (IN)

(21) Appl. No.: 18/548,222

(22) PCT Filed: Jun. 28, 2023
(86) PCT No.: PCT/IN2023/050630

§ 371 Date: Aug. 29, 2023

(30) Foreign Application Priority Data
Apr. 28, 2023 (IN) 202341030784

(51) Int. Cl. A61K 31/4375; A61K 9/00; A61K 36/74; A61K 47/10; A61K 47/12; A61K 47/26; A61K 47/36; A61K 47/38; A61K 47/46

(52) U.S. Cl. CPC A61K 31/4375; A61K 9/006; A61K 36/74; A61K 47/10; A61K 47/12; A61K 47/26; A61K 47/36; A61K 47/38; A61K 47/46

(57) ABSTRACT

The invention relates to oral dispersible film of Mitragynine. More particularly, the invention relates to a rapidly dispersible, fast acting oral dispersible film of Mitragynine comprising purified Mitragynine free base or purified Mitragynine salts. The invention also relates to a process for preparing such oral dispersible film of Mitragynine.

### CROSS-REFERENCE TO RELATED APPLICATION(S)

This application claims the priority to, and benefit of Indian Complete Patent Application No. 202341030784 filed on Apr. 28, 2023; the disclosures of which are incorporated herein by reference.

### FIELD OF THE INVENTION

The invention relates to oral dispersible film of Mitragynine. More particularly, the invention relates to a rapidly dispersible, fast acting oral dispersible film of Mitragynine comprising purified Mitragynine free base or Mitragynine salts. The invention also relates to a process for preparing such oral dispersible film of Mitragynine.

### BACKGROUND OF THE INVENTION

Novel drug delivery has been promising field which has been developed to address the challenges faced in conventional drug delivery system. Novel drug delivery includes transdermal drug delivery system, nanoparticles, liposomes, microencapsulation, controlled drug delivery system, and oral dispersible drug.

Various drug delivery systems have been introduced in the recent days in the field of pharmaceutical technology. Transdermal patch is a technique from which a new drug delivery system that is oral dispersible film was developed. Oral dispersible films when placed in mouth or oral cavity, they disintegrate or dissolve within a minute. Further to this, it does not require chewing or drinking of water, which enhances the patient compliance. There are number of ingredients that are used to formulate oral dispersible patches, like active pharmaceutical ingredients, polymers, flavouring agents, colouring agents etc, in which polymer acts as important ingredient which helps to form the film. Usually, hydrophobic polymers are used for oral dissolving films which breaks down on the tongue or buccal cavity and then drug will go to systemic circulation. Fast disintegrating property, good mechanical property like mouth dissolving property of film can be achieved by water soluble polymer. Moreover, in oral dispersible films, accurate and effective dosing can be accomplished in a safe manner.

Oral dispersible films have been introduced in the market as an alternative to conventional oral dosage forms to enhance patient compliance. For example, mucoadhesive film formulations have been described that improve absorption of pharmaceutical agents through the mucosal tissue to bypass barriers in the gastrointestinal tract. Oral dispersible films also can overcome the swallowing problems associated with the capsules or tablets. The limitations of conventional drug delivery system like frequent administration, an increased chance of missing in administering desired drug dose, slow onset of action, high first pass metabolism, have been overcome by recent advancement in oral dispersible films.

The advantage of oral dispersible drug delivery system lies in its ability to dissolve in the mouth and quickly disintegrate in saliva compared to conventional oral dosage forms. As the drug dissolves faster, the therapeutic effect of the drug is quicker. Orally disintegrating dosage forms are becoming increasingly important in pharmaceutical market across the globe, as well as in over-the-counter drugs. Oral disintegrating dosage forms are patient centric drug delivery system wherein they are designed to increase the compliance and bioavailability of the drug.

Oral dispersible drug delivery system increases patient compliance as they are easy to swallow and do not require water to consume.

There are 3 subtypes of oral quick dissolving films:
1. Rapid release film
2. Mucoadhesive melt-away wafer
3. Mucoadhesive sustained release wafers.

As several physicochemical properties should be considered during the preparation of composition, the major aspect to be considered during formulation is quick disintegration of drug/active ingredient as soon as it is administered into mouth.

Thus, an easy method of delivering oral formulations has been found to be in the form of rapid dissolving single or multi-layer film carrying active ingredient in the formulation. Since the primary use of all thin film oral dosage form relies on their disintegration in the saliva of the oral cavity, the final film that is used must preferably be water soluble. Prior arts reveal that, oral dispersible films are of two types:
a. orally disintegrating films, and
b. orally dissolving films

Orally disintegrating films are poorly soluble in water, disintegrates in mouth followed by dissolution and absorption in gastrointestinal tract. On the other hand, orally dissolving films are water soluble and thus disintegrates and dissolves in mouth. Due to the escape of first pass metabolism, this delivery system provides faster action.

Mitragyna speciosa, is a tropical evergreen tree. It belongs to the plant family of Rubiaceae. It is majorly found in southeast Asian countries. It was first isolated in 1921 and its chemical structure was fully elucidated in 1964. It has been used as an herbal drug for a long time. It is commonly known as kratom. Typically, kratom leaves were used for chewing. Dry kratom can be swallowed or brewed. At a low dose, kratom is used to reduce fatigue, as it causes euphoric effect. The effect usually begins within 5 to 10 minutes. There are more than 50 structurally related alkaloids which can be isolated from various parts of the plant. Mitragynine, Corynantheidine, 7-hydroxamitragynine are three major alkaloids present in extraction of Mitragyna speciosa.

Mitragyna speciosa is consumed in the form of direct leaves, extract in water, swallowing capsules, as a green tea, and powder adding to daily meal or snacks or milk shake. In general, Mitragyna speciose crude extract as per the available literature is between 2 and 8 grams.

Till date there are no defined therapeutic dose of Mitragyna speciosa that is proved through an approved clinical route. Mitragyna speciosa is traditionally used in different doses for different purposes in different dosage forms.

However, there has not been any research or limited research on formulating a composition of Mitragyna speciosa extract especially Mitragynine that can quickly disperse in mouth.

WO 2018127938 discloses an oral dispersible film composition comprising maltodextrin and Hydroxypropyl cellulose, polyethylene glycol, or its combination with triethyl citrate, propylene glycol. However, said prior art fails to disclose Mitragyna speciosa extract such as Mitragynine as active agent in oral composition.

WO2016009001A1 discloses an oro-dispersible film comprising suspension comprising a plant extract. The film forming polymer has been selected from cellulose, cellulose ester, cellulose ether, oxidized polyethylene, polyvinyl pyrrolidone, polyvinyl alcohol, pullulan, starch, modified starch, gelatine, pectin, alginate, and their combination. Microcrystalline cellulose has been used as a suspension. Nevertheless, this invention fails to disclose the Mitragyna speciosa extract oral composition or its delivery at different concentrations of the plant extract.

US20220024923A1 reveals Mitragynine analogues and pharmaceutical salts thereof. The compounds provided may have both agonistic and antagonistic effect on one or more opioid receptors. It also discloses the methods of using the compounds for treating or managing pain. However, this prior art fails to disclose the oral dispersible film composition of Mitragyna speciosa extract. It also fails to reveal the process involved in the production of oral dispersible film composition including the various studies involved in the preparation.

Therefore, there exists a need to formulate an oral dispersible film composition of Mitragyna speciosa extract particularly purified Mitragynine free base or purified Mitragynine salts which addresses challenges like active ingredient interference with film formation, film stability and desirable film properties like taste, endurance, palatability, thickness, and intolerance to humidity without compromising disintegration time, texture, and appearance.

### OBJECTS OF THE INVENTION

The primary object of the present invention is to provide an oral dispersible film of Mitragynine.

Another object of the invention is to provide a rapidly dispersible, fast acting oral dispersible film of Mitragynine.

Another object of the invention is to provide an oral dispersible film comprising purified Mitragynine free base or purified Mitragynine salts.

Yet another object of the present invention is to minimise the challenges faced by oral dispersible film such as film formation, film stability, and desirable film properties like taste, endurance, palatability, thickness, and intolerance to humidity.

A further object of the invention is to provide a process for preparing oral dispersible film of Mitragynine.

### SUMMARY OF THE INVENTION

Accordingly, the present invention provides an oral dispersible film of Mitragynine comprising:
i. Purified Mitragynine;
ii. One or more film forming polymers;
iii. One of more disintegrants;
iv. One of more Stabilizers; and
v. Optionally, one or more flavouring agents.

Wherein, the purified Mitragynine is Mitragynine free base or Mitragynine salt.

In one embodiment, the purified Mitragynine is Mitragynine free base. The Mitragynine free base may be in crystalline form or amorphous form. Preferably, the Mitragynine free base is in amorphous form.

In the oral dispersible film of the present invention, the purified Mitragynine free base has 10-99% purity by HPLC.

In another embodiment, the purified Mitragynine is Mitragynine salt. The purified Mitragynine salt is selected from Mitragynine hydrochloride salt, purified Mitragynine hydrobromide salt, purified Mitragynine perchlorate salt, purified Mitragynine sulphate salt, purified Mitragynine oxalate salt, purified Mitragynine tartrate salt, purified Mitragynine citrate salt, purified Mitragynine formate salt, purified Mitragynine trifluoro acetate salt, or purified Mitragynine trichloro acetate salt. Preferably, the purified Mitragynine salt is purified Mitragynine oxalate salt.

In the oral dispersible film of the present invention, the purified Mitragynine salt has 10-99% purity by HPLC.

In the oral dispersible film of the present invention, the purified Mitragynine is present at a concentration of 5-200 mg per film.

The film forming polymer in the said oral dispersible film is selected from the group comprising of polyvinyl alcohol (PVA), polyethylene glycol, hydroxyl methyl cellulose, hydroxypropylmethyl cellulose (HPMC), chitin, pectin, gelatine, chitosan, gaur gum, gum kaya, fenugreek seed mucilage, Soy polysaccharide, pullulan, or combination thereof.

Preferably the film forming polymer is selected from hydroxypropylmethyl cellulose (HPMC), polyvinyl alcohol (PVA), pullulan or combination thereof.

The film forming polymer in the said oral dispersible film is present at a concentration of 10-100 mg per film.

The disintegrants in the said oral dispersible film are polyhydric alcohols selected from the group comprising of sorbitol, mannitol, isomalt, maltitol, or combination thereof, preferably the disintegrant is mannitol.

In the said oral dispersible film, the disintegrant is present at a concentration of 1-100 mg per film.

The stabilizers employed in the said oral dispersible film are selected from maltodextrin, mannitol, glucose, lactose, dextrose, lactitol, erythrol or xylitol.

Preferably, the stabilizer is maltodextrin.

In the said oral dispersible film the stabilizer is present at a concentration of 0.5-50 mg per film.

The flavouring agents in the said oral dispersible film are selected from cherry, peppermint, mango, ginger, clove, spearmint, coffee with mint.

The said oral dispersible film further comprises of preservatives, sweetening agents, and plasticizers.

The preservatives employed in the said oral dispersible film are selected from sorbic acid/potassium sorbate, methyl-4-hydroxybenzoate and propyl-4-hydroxybenzoate, preferably the preservative is Sodium benzoate.

In the said oral dispersible film the preservative is present at a concentration of 1-50 mg per film.

The sweetening agents employed in the said oral dispersible film are selected from the group comprising of mannitol, glucose, lactose, dextrose, lacitol, erythrol, xylitol, aspartame, neotame, sodium saccharin, or sucralose, preferably the sweetening agent is sucralose.

In the said oral dispersible film the sweetening agent is present at a concentration of 1-50 mg per film.

The plasticiser employed in the said oral dispersible film is glycerine, which present at a concentration of 1-100 mg per film.

In the said oral dispersible film of the present invention, water is added as a bulk medium.

The said oral dispersible film of the present invention has a thickness of 0.6-0.8 mm and wherein the disintegration time of the film is between 30-60 seconds.

In another aspect, present invention discloses an oral dispersible film of Mitragynine, comprises:
i. Purified Mitragynine oxalate salt: 5-200 mg/film;
ii. Hydroxypropylmethyl cellulose (HPMC) and pullulan: 10-100 mg/film;
iii. Mannitol: 1-100 mg/film;
iv. Maltodextrin: 0.5-50 mg/film;
v. Flavouring agent selected from cherry, peppermint, mango, ginger, clove, spearmint: 1-10 mg/film;
vi. Sodium benzoate: 1-50 mg/film;
vii. Sucralose: 1-50 mg/film;
viii Glycerine: 1-100 mg/film; and
ix. Water: q.s.

The said oral dispersible film has:
Thickness of film is 0.6-0.8 mm
Endurance is 3-4 kp
pH value: 2.0 to 3.2
Disintegration time is 50-60 sec.

In the oral dispersible film of the present invention, purified Mitragynine is obtained from Mitragyna speciosa.

In another aspect, present invention discloses a process for preparation of oral dispersible thin film of purified Mitragynine, comprising the following steps of:
a. mixing disintegrant, stabilizer, preservative, sweetening agent and plasticizer in 50% water, until a clear solution is formed;
b. adding the film forming polymer to the clear solution of step (a) followed by mixing to obtain a homogeneous mixture;
c. checking for lumps or insoluble material followed by mixing to obtain a clear solution;
d. uniformly mixing purified Mitragynine in 50% water, until a uniform dispersion is formed;
e. mixing clear solution of step (c) and uniform dispersion of Mitragynine of step (d) to obtain a uniform viscous solution;
f. adding flavouring agent to the uniform viscous solution of step (e) followed by mixing; and
g. coating and drying the viscous solution of step (f) to form thin oral dispersible film followed by packaging.

Wherein, the purified Mitragynine is Mitragynine free base or Mitragynine salt.

In one embodiment, the purified Mitragynine is Mitragynine free base. The Mitragynine free base may be in crystalline form or amorphous form. Preferably, the Mitragynine free base is in amorphous form.

In the said process the purified Mitragynine free base has 10-99% purity by HPLC.

In another embodiment, the purified Mitragynine is Mitragynine salt. The purified Mitragynine salt is selected from Mitragynine hydrochloride salt, purified Mitragynine hydrobromide salt, purified Mitragynine perchlorate salt, purified Mitragynine sulphate salt, purified Mitragynine oxalate salt, purified Mitragynine tartrate salt, purified Mitragynine citrate salt, purified Mitragynine formate salt, purified Mitragynine trifluoro acetate salt, or purified Mitragynine trichloro acetate salt. Preferably, the purified Mitragynine salt is purified Mitragynine oxalate salt.

In the said process, the purified Mitragynine salt has 10-99% purity by HPLC.

In the said process, the mixing is carried out with overhead stirrer.

In the said process drying process is selected from spray drying, vacuum drying, freeze drying, tray drying, rotatory drying, drum drying, or fluidized bed drying.

The purified Mitragynine is obtained from Mitragyna speciosa.

A new therapeutic route of administration of Mitragynine has been developed which will help patient administration to become easy and fast-acting. Accordingly, the present inventors have developed a rapidly dispersible, fast acting oral dispersible film of Mitragynine comprising purified Mitragynine free base or Mitragynine salts.

### DETAILED DESCRIPTION OF THE INVENTION

The following detailed description refers to the specific embodiments in which the invention may be practised. These embodiments are described in sufficient detail to enable those skilled in the art to practise the invention. Other embodiments may be utilized, and changes may be made without departing from the scope of the invention. The various embodiments are not necessarily mutually exclusive, as some embodiments can be combined with one or more other embodiments to form new embodiments.

Unless defined otherwise, all technical and scientific terms used herein have the same meaning as commonly understood by one of ordinary skill in the art to which the methods belong.

Further the embodiments described herein can be understood more readily by reference to the following detailed description and examples. Methods described herein are merely illustrative of the principles of the present invention and are not limited to the specific embodiments presented in the detailed description and examples. Numerous modifications and adaptations will be readily apparent to those of skill in the art without departing from the spirit and scope of the invention.

Where a range of values is provided, it is understood that each intervening value between the upper and lower limit of that range and any other stated or intervening value in that stated range, is encompassed within by the methods. The upper and lower limits of these smaller ranges may independently be included in the smaller ranges and are also encompassed within by the methods, subject to any specifically excluded limit in the stated range. Where the stated range includes one or both of the limits, ranges excluding either or both of those included limits are also included in the methods.

It is known that oral dispersible films comprising a plant extract are unwieldly to manufacture due to the nature of the plant extracts. Plant extracts are generally in an oil form, concentrated solution, or dry form. They are composed of many phytochemical constituents, which are present in crude form. Thus, plant extracts and other active ingredients are not present in the form of crystalline solids compounds, nor they are in the form of nanoparticles.

The oral dispersible films of the present invention typically can carry and deliver Mitragyna speciosa extract at different concentrations. The current invention addresses a common challenges like active ingredient interfere with film formation, film stability and desirable film properties like taste, endurance, palatability, thickness, and intolerance to humidity without compromising disintegration time, texture, and appearance.

The Film of the present invention, which is being coated on the dosage form is an edible film, it is also called as mouth dissolving film. It is used to deliver the desired drug systematically to achieve the therapeutic and pharmacological effect. The quick disintegration without requiring water, and quick pharmacological action, makes increase in demand for oral dispersible forms.

The present invention addresses the challenges faced in active ingredient interfere with the film formation, film formation refers to the process of forming a thin film on surface, before the surface has been coated with the active drug substance.

Accordingly, the present invention relates to an oral dispersible film containing Mitragyna speciosa plant extract in an acceptable form. Preferably the acceptable form is purified Mitragynine, wherein the purified Mitragynine is Mitragynine free base or Mitragynine salt.

The present invention relates to an oral dispersible film comprising purified Mitragynine and its salts thereof and a film forming polymer which delivers the desired drug at varied concentrations.

The present disclosure relates to an edible film comprising (i) a film-forming polymer, (ii) a disintegrant, and (iii) a stabilizer.

Film-forming polymer is one or both of polyvinyl alcohol (PVA) or Pullulan (α-1,4-; α-1,6-glucan) and hydroxypropyl methyl cellulose (HPMC). In certain embodiments, the film comprises only one of PVA or HPMC.

In certain embodiments, the film forming polymer is not hydrophilic. In certain embodiments, the film forming polymer is hydrophobic. In certain embodiments, the film further includes a plasticizer. In some embodiments, the film comprises one or more film forming polymer dispersants. In any of the embodiments, the film forming polymer comprises one or more active agents.

Accordingly in one aspect present invention discloses an oral dispersible film of Mitragynine comprising:
i. Purified Mitragynine;
ii. One or more film forming polymers;
iii. One of more disintegrants;
iv. One of more stabilizers;
v. Optionally, one or more flavouring agents.

In the said composition, the purified Mitragynine is Mitragynine free base or Mitragynine salt.

In one embodiment, the purified Mitragynine is Mitragynine free base. The Mitragynine free base present in the instant invention is in crystalline form or amorphous form. Preferably, the Mitragynine free base is in amorphous form.

In the oral dispersible film of the present invention, the purified Mitragynine free base has 10-99% purity by HPLC. Preferably, the purity of Mitragynine free base is 50% by HPLC.

In another embodiment, the purified Mitragynine is Mitragynine salt. The purified Mitragynine salt is selected from Mitragynine hydrochloride salt, purified Mitragynine hydrobromide salt, purified Mitragynine perchlorate salt, purified Mitragynine sulphate salt, purified Mitragynine oxalate salt, purified Mitragynine tartrate salt, purified Mitragynine citrate salt, purified Mitragynine formate salt, purified Mitragynine trifluoro acetate salt, or purified Mitragynine trichloro acetate salt. Preferably, the purified Mitragynine salt is purified Mitragynine oxalate salt.

In the oral dispersible film of the present invention, the purified Mitragynine salt has 10-99% purity by HPLC. Preferably, the purity of Mitragynine salt is 75% by HPLC.

The purified Mitragynine is present in the said oral dispersible film at a concentration of 50-200 mg per film.

In the present oral dispersible film, the film forming polymer is selected from the group of synthetic polymers like polyvinyl alcohol, polyethylene glycol, hydroxyl methyl cellulose and hydroxypropylmethyl cellulose (HPMC). In some embodiment, the film forming polymer is selected from natural sources like chitin, pectin, gelatine, chitosan, gaur gum, gum kaya, fenugreek seed mucilage, Soy polysaccharide, pullulan etc.

In some embodiments, the film forming polymer is water soluble polymer or water insoluble polymer with low molecular weight and excellent film forming capacity.

Preferably, the film forming polymer is selected from hydroxypropylmethyl cellulose (HPMC), polyvinyl alcohol (PVA), pullulan or combination thereof.

In the said oral dispersible film, the film forming polymer is present at a concentration of 10-100 mg per film.

In some embodiment, disintegrants employed in present oral dispersible film are polyhydric alcohols selected from group comprising of sorbitol, mannitol, isomalt, and maltitol, which are used either alone or in combinations. These disintegrates provide good mouth fell and cooling sensation when dissolved in the oral cavity. These disintegrants could also be used as either as natural sweetening agent or artificial sweetening agents.

The disintegrants are present at a concentration of 1-100 mg per oral dispersible film.

In some embodiment, stabilizers employed in present oral dispersible film are selected from maltodextrin, mannitol, glucose, lactose, dextrose, lactitol, erythrol or xylitol. Stabilizers in the said film prevents unwanted change in the state of the oral dispersible film. Preferably the stabilizer employed in present oral dispersible film is maltodextrin.

The stabilizer is present at a concentration of 0.5-50 mg per oral dispersible film.

The Flavouring agents can be optionally added to oral dispersible film to increase the patient's compliance, improve elegance, mask unpleasant taste, complement colour, etc. The flavouring agents added in the said oral dispersible films are cherry, peppermint, mango, ginger, clove, spearmint, coffee with mint etc.

In some embodiment, the oral dispersible film additionally comprises preservatives, sweetening agents, and plasticizers.

Preservative is used to prolong shelf-life and enhance the activity of the desired substance. Usually, antifungal preservative or antibacterial preservatives are employed in the composition. Preservative employed in the present oral dispersible films are selected from sorbic acid/potassium sorbate, methyl-4-hydroxybenzoate and propyl-4-hydroxybenzoate. Sodium benzoate is preferred preservatives in an oral edible dispersible film composition of the present invention. Preservatives are added from quantity ranging from 1-50 mg per film.

Sweetening agents are the chemical substances which are added to either mask the unpleasant taste or enhance the perception of sweet taste in thin films. In some embodiments, natural sweetening agents like mannitol, glucose, lactose, dextrose, lacitol, erythrol, xylitol are being used. Artificial sweetening agents like aspartame, neotame, sodium saccharin, sucralose are also used. In present oral dispersible film, sucralose is the preferred sweetening agent.

The quantity of sweetening agents in the present film ranges between 1-50 mg per film.

Plasticizer helps in improving the flexibility of the film and reduces the brittleness of the film. Plasticizer also needs to be compatible with polymer and solvent as it also enhances the strength of polymer. Plasticiser preferred is present film is glycerine, selected from the range of 1-100 mg per film. The role of plasticizer plays a vital role in fast delivery of the drug.

In present embodiment water is used as the bulk medium to dissolve water soluble polymers, sweeteners, stabilizers, preservatives, and other ingredients. The present composition comprises about 200 to 450 ml of water.

The present invention also discloses an oral dispersible film of Mitragynine, comprising:
i. Purified Mitragynine oxalate salt: 5-200 mg/film;
ii. Hydroxypropylmethyl cellulose (HPMC) and pullulan: 10-100 mg/film;
iii. Mannitol: 1-100 mg/film;
iv. Maltodextrin: 0.5-50 mg/film;
v. Flavouring agent selected from cherry, peppermint, mango, ginger, clove, spearmint: 1-10 mg/film;
vi. Sodium benzoate: 1-50 mg/film;
vii. Sucralose: 1-50 mg/film;
viii. Glycerine: 1-100 mg/film; and
ix. Water: q.s.

According to the present invention, purified Mitragynine is obtained from Mitragyna speciosa.

To determine the quality of present oral dispersible film, following are the components of the stability studies considered:
a) Thickness of film: The thickness measured is an important aspect of an oral dispersible film, as it is directly related to the quantity of the active pharmaceutical ingredients present. The thickness ensures the robustness of the film. At the same time, it shall also be in accordance with the criteria of patient compliance. The standard thickness of the film should be about 0.3-1.3 mm.
b) Endurance: Endurance studies reveals the folding capacity of the film. Folding endurance is generally carried out by repeated folding of the film at the same place till the film is broken. The number of folds before the break is considered as the endurance value.
c) Determination of pH value: Determining pH is also an important parameter to be considered for the stability studies of oral dispersible film. pH determines taste properties, rapid release of the drug, dispersible time.
d) Disintegration time: Disintegration time is the time required to disintegrate the film when it is brought in contact with water or the saliva. It is generally dependent on weight and thickness of the film. Disintegration time is analysed by a disintegration test apparatus, which is being prescribed by various pharmacopeia across the globe. Normally disintegration time ranges from 50-60 seconds for a fast-dissolving film.

In another aspect present invention discloses a process for manufacturing oral dispersible thin film of purified Mitragynine.

Following are the methods usually used in manufacturing of oral dispersible thin film, that are generally known in the art:
a. Solvent casting method: Solvent casting method is generally preferred method in manufacturing oral dispersible films, because of its simple preparation, economical, and easy for applications. Few advantages of using solvent casting method are films are uniform thickness, better physical properties, flexible film.
b. Hot melt extrusion method: The mixture containing formulation component are mixed and melted by means of a heater, thus a liquid mixture is turned into a thin film.
c. Semi solid method: In this method, water soluble polymers are dissolved in water and further added to the solution of acid insoluble polymer where the semisolid consistency is obtained, further processed for sonication it is coated on non-treated casting film.
d. Solid dispersion extrusion: Solid dispersion is prepared by extruding the formulation components, with active pharmaceutical ingredients and further made into thin film.
e. Rolling: A solution or suspension containing the active pharmaceutical ingredient is rolled over a carrier and further process is carried out.

Accordingly, present invention discloses a process for preparation of oral dispersible thin film of purified Mitragynine, comprising the following steps of:
a. mixing disintegrant, stabilizer, preservative, sweetening agent and plasticizer in 50% water, until a clear solution is formed;
b. adding the film forming polymer to the clear solution of step (a) followed by mixing to obtain a homogeneous mixture;
c. checking for lumps or insoluble material followed by mixing to obtain a clear solution;
d. uniformly mixing purified Mitragynine in 50% water, until a uniform dispersion is formed;
e. mixing clear solution of step (c) and uniform dispersion of Mitragynine of step (d) to obtain a uniform viscous solution;
f. adding flavouring agent to the uniform viscous solution of step (e) followed by mixing; and
g. coating and drying the viscous solution of step (f) to form thin oral dispersible film followed by packaging.

### Step (a)

Step (a) includes mixing of disintegrant, stabilizer, preservative, sweetening agent, and plasticizer in 50% water. Mixing continues until a clear solution is formed.

Mixing is done to improve dissolution and diffusion of constituents of mixture to interact with each other. Mixing of all the excipients could be done by various mixing methods like high-speed mixer, batch mixer, V type mixer, electric mixer, overhead stirring, sigma mixer, turbine mixer, sonic and ultrasonic mixer. Preferably, mixing is carried out with overhead stirrer.

In the present process water is used as a bulk medium. Out of the total amount of water, 50% water is used in this step to dissolve disintegrant, stabilizer, preservative, sweetening agent, and plasticizer.

Disintegrants employed in present process are polyhydric alcohols selected from group comprising of sorbitol, mannitol, isomalt, maltitol, or combination thereof. Preferably the disintegrant is mannitol.

Stabilizers employed in present process are selected from maltodextrin, mannitol, glucose, lactose, dextrose, lactitol, erythrol or xylitol. Preferably the stabilizer is maltodextrin.

Preservatives employed in present process are selected from sorbic acid/potassium sorbate, methyl-4-hydroxybenzoate and propyl-4-hydroxybenzoate. Sodium benzoate is preferred preservative.

Sweetening agents employed in present process is selected from mannitol, glucose, lactose, dextrose, lacitol, erythrol, aspartame, neotame, sodium saccharin, or sucralose. Sucralose is the preferred sweetening agent in the present process.

Plasticiser preferred is present process is glycerine.

### Step (b)

The film forming polymer employed in step (b) is selected from the group of synthetic polymers like polyvinyl alcohol, polyethylene glycol, hydroxyl methyl cellulose and hydroxypropylmethyl cellulose (HPMC). In some embodiment, the film forming polymer is selected from natural sources like chitin, pectin, gelatine, chitosan, gaur gum, gum kaya, fenugreek seed mucilage, Soy polysaccharide, pullulan etc.

Preferably, the film forming polymer is selected from hydroxypropylmethyl cellulose (HPMC), polyvinyl alcohol (PVA), pullulan or combination thereof.

Said film forming polymer is added to the clear solution of step (a) followed by mixing to obtain a homogeneous mixture.

### Step (c)

In step (c) the mixing is carried out using an overhead stirrer to obtain a clear solution.

### Step (d)

Step (d) includes mixing of purified Mitragynine in 50% water, until a uniform dispersion is formed.

The purified Mitragynine is dissolved in remaining 50% water to obtain a uniform dispersion. The purified Mitragynine employed in the said process is Mitragynine free base or Mitragynine salt.

In one embodiment, the purified Mitragynine is Mitragynine free base. The Mitragynine free base present in the instant invention is in crystalline form or amorphous form. Preferably, the Mitragynine free base is in amorphous form.

The purified Mitragynine free base has 10-99% purity by HPLC. Preferably, the purity of Mitragynine free base is 50% by HPLC.

In another embodiment, the purified Mitragynine is Mitragynine salt. The purified Mitragynine salt is selected from Mitragynine hydrochloride salt, purified Mitragynine hydrobromide salt, purified Mitragynine perchlorate salt, purified Mitragynine sulphate salt, purified Mitragynine oxalate salt, purified Mitragynine tartrate salt, purified Mitragynine citrate salt, purified Mitragynine formate salt, purified Mitragynine trifluoro acetate salt, or purified Mitragynine trichloro acetate salt. Preferably, the purified Mitragynine salt is purified Mitragynine oxalate salt.

The purified Mitragynine salt bas 10-99% purity by HPLC. Preferably, the purity of Mitragynine salt is 75% by HPLC.

The said purified Mitragynine is obtained from Mitragyna speciosa.

### Step (e)

Mixing clear solution of step (c) and uniform dispersion of Mitragynine of step (d) with overhead stirrer to obtain a uniform viscous solution.

### Step (f)

The flavouring agents added in the said process are cherry, peppermint, mango, ginger, clove, spearmint, coffee with mint etc.

### Step (g)

In some embodiments drying process is selected from various processes like spray drying, vacuum drying, freeze drying, tray drying, rotatory drying, drum drying, fluidized bed drying. Spray drying process is preferred in drying oral dispersible films.

The obtained thin oral dispersible film is packed. Usually, packaging is done by strip packing, blister packaging, aluminium foil packaging, accu-counter tablet packing, automatic pouch packing. Strip packing is preferred in oral dispersible films.

All the steps are performed at room temperature.

Present invention provides an easy method of delivering oral formulations of Mitragynine in the form of rapid dissolving film. The present oral dispersible film is developed in such a way that the taste of Mitragyna masks without feel. Since the film is soluble and absorbs rapidly in gut, it shows high bioavailability or efficacy when compared to equivalent crude extract consumption. This route of administration is also considered to be safer, as dose is less and not influencing digestive track massively.

The oral dispersible film of the invention can be used as quick dissolving, fast acting therapy for varied types of pain management. The oral dispersible film of the invention can be used to alleviate moderate-to severe acute pain, as an effective analgesic for many types of pain, including but not limited to paroxysmal spontaneous pain, steady pain, allodynia associated with postherpetic neuralgia. It can be effectively used in management of cancer-related and chronic pain. The oral dispersible film of the invention can also be used in therapeutic dosage in improving quality of life of patients with many types of pain, providing them quick energy, helping in focus, reducing anxiety and depression etc.

The present oral dispersible thin film is the new therapeutic route of administration of Mitragynine which will help patient administration to become easy and fast-acting.

Certain specific aspect and embodiment of the present invention will be explained in detail with reference to the following examples, which are provided only for purposes of illustration and should not be construed as limiting the scope of the invention in any manner.

### EXAMPLES

The purified Mitragynine Free base or purified Mitragynine salt may be obtained by the process disclosed in Indian patent application Nos. IN202341010930; IN202341010938 or IN202341010947, all three filed by the applicant in Indian Patent Office on Feb. 17, 2023, or any other process known in the art.

Particularly, the purified Mitragynine Free base may be obtained by the process disclosed in Indian Patent Application No. IN202341010930, or any other method known in the art.

More particularly, the purified salt of Mitragynine (purified Mitragynine oxalate salt) with purity more than 75% was obtained by the improved process for purification of Mitragynine from Mitragyna speciosa plant, which is developed by the present inventor and is disclosed and filed separately in another Indian patent application No. IN202341010947 filed in Indian Patent Office, on Feb. 17, 2023. Or it may be obtained by any other process known in the art.

The plant material for extraction of the Mitragynine extract was obtained from commercial vendors/suppliers in Indonesia and Thailand.

The Mitragynine extract was obtained from the plant material by a process disclosed in Indian Patent Application No. 202341010687 filed by the applicant in Indian Patent Office on Feb. 17, 2023, or Mitragynine extract may be obtained by any other process known in the art.

### Example 1: Generalized Oral Dispersible Film Components

The Oral dispersible film is prepared according to following concentration range of the ingredients along with role of each ingredient used is mentioned in below table:

TABLE 1
General composition of Oral dispersible film and their role

| Ingredients along with its Role | Mg/film |
|---|---|
| Active Pharmaceutical Ingredient-Purified | 5-200 |
| Mitragynine oxalate salt | |
| Film forming polymers-selected from | 30-100 |
| hydroxypropylmethyl cellulose (HPMC), | |
| polyvinyl alcohol (PVA), pullulan or | |
| combination thereof | |
| Disintegrant-Mannitol | 1-20 |
| Stabilizers-Maltodextrin | 0.5-20 |
| Flavouring agents selected from cherry, | 1-5 |
| peppermint, mango, ginger, clove, spearmint | |
| Preservative-Sodium benzoate | 1-50 |
| Sweetening agent-Sucralose | 1-50 |
| Plasticiser-Glycerine | 1-100 |
| Water | q.s. |

### Example 2: Oral Dispersible Film-Ingredients and Concentration of Said Composition

| Sr. No. | Ingredients | mg/film |
|---|---|---|
| 1 | Purified Mitragynine oxalate salt | 100 |
| | 75% Purity | |
| 2 | Hydroxypropylmethyl cellulose (HPMC) | 50 |
| 3 | Pullulan | 20 |
| 4 | Mannitol | 10 |
| 5 | Maltodextrin | 15 |
| 6 | Coffee with mint flavour | 3 |
| 7 | Sodium benzoate | 4 |
| 8 | Sucralose | 13 |
| 9 | Glycerine | 40 |
| 10 | Water | 400 |

### Example 3: Process for Preparation of Oral Dispersible Film

Water was used as a bulk medium, wherein out of the total amount of water, 50% water was used to dissolve Disintegrant—Mannitol, Stabilizers—Maltodextrin, Preservative—Sodium benzoate, Sweetening agent—Sucralose and Plasticiser—Glycerine. All the above raw materials along with 50% water was added to reactor and mixed with overhead stirrer to obtain a clear solution. To this clear solution film forming polymers—Hydroxypropylmethyl cellulose (HPMC) and Pullulan were added followed by mixing with overhead stirrer to obtain a homogeneous mixture.

The above homogeneous mixture comprising all the excipients were checked for any lumps or insoluble material followed by mixing with overhead stirrer to obtain a homogeneous clear solution.

The Purified Mitragynine oxalate salt (75% Purity) was mixed with remaining 50% water in another reactor with overhead stirrer to obtain uniform dispersion of Mitragynine salt.

The above obtained uniform dispersion of Mitragynine salt was added to the homogeneous clear solution of the excipients obtained above in another reactor. The said solution is vigorously mixed to obtain a uniform viscous solution. To this viscous solution flavouring agent was added with continued mixing.

The obtained uniform viscous solution was coated and dried to form thin oral dispersible film followed by packaging.

Coating indicates, forming a thin film on the PET membrane to obtain a final film.

The obtained film was cut into required sizes (30×30 mm). This size carries or hold 50 mg of active ingredient.

All the steps are performed at room temperature.

### Example 4: Physico-Chemical Parameters of Obtained Oral Dispersible Film

The Thickness of film, Endurance, pH value and Disintegration time of the Oral dispersible film was studied.

### Methodology Used:

a) Thickness of film: By vernier calliper
b) Endurance: Repeated folding techniques
c) Determination of pH value: pH Meter
e) Disintegration time: By using disintegration tester (Make: Electrolab)

### Results of Physicochemical Parameters Analysis:

| Parameters | Values |
|---|---|
| Thickness of film | 0.6-0.8 mm |
| Endurance | 3-4 kp |
| pH value | 2.0 to 3.2 |
| Disintegration time | 50-60 sec |

1. An oral dispersible film of Mitragynine comprising:
i. Purified Mitragynine;
ii. One or more film forming polymers;
iii. One of more disintegrants;
iv. One of more stabilizers; and
v. Optionally, one or more flavouring agents.

2. The oral dispersible film as claimed in claim 1, wherein the purified Mitragynine is Mitragynine free base or Mitragynine salt.

3. The oral dispersible film as claimed in claim 2, wherein the Mitragynine free base is in crystalline or amorphous form.

4. The oral dispersible film as claimed in claim 3, wherein the Mitragynine free base is in amorphous form.

5. The oral dispersible film as claimed in claim 2, wherein the purity of Mitragynine free base is 10-99% by HPLC.

6. The oral dispersible film as claimed in claim 2, wherein the purified Mitragynine salt is selected from purified Mitragynine hydrochloride salt, purified Mitragynine hydrobromide salt, purified Mitragynine perchlorate salt, purified Mitragynine sulphate salt, purified Mitragynine oxalate salt, purified Mitragynine tartrate salt, purified Mitragynine citrate salt, purified Mitragynine formate salt, purified Mitragynine trifluoro acetate salt, or purified Mitragynine trichloro acetate salt.

7. The oral dispersible film as claimed in claim 6, wherein the purified Mitragynine salt is purified Mitragynine oxalate salt.

8. The oral dispersible film as claimed in claim 2, wherein the purity of Mitragynine salt is 10-99% by HPLC.

9. The oral dispersible film as claimed in claim 1, wherein the purified Mitragynine is present at a concentration of 5-200 mg per film.

10. The oral dispersible film as claimed in claim 1, wherein the film forming polymer is selected from the group comprising polyvinyl alcohol (PVA), polyethylene glycol, hydroxyl methyl cellulose, hydroxypropylmethyl cellulose (HPMC), chitin, pectin, gelatine, chitosan, gaur gum, gum kaya, fenugreek seed mucilage, Soy polysaccharide, pullulan, or combination thereof.

11. The oral dispersible film as claimed in claim 10, wherein the film forming polymer is selected from hydroxypropylmethyl cellulose (HPMC), polyvinyl alcohol (PVA), pullulan or combination thereof.

12. The oral dispersible film as claimed in claim 1, wherein the film forming polymer is present at a concentration of 10-100 mg per film.

13. The oral dispersible film as claimed in claim 1, wherein the disintegrants are polyhydric alcohols selected from the group comprising of sorbitol, mannitol, isomalt, maltitol, or combination thereof.

14. The oral dispersible film as claimed in claim 13, wherein the disintegrant is mannitol.

15. The oral dispersible film as claimed in claim 1, wherein the disintegrant is present at a concentration of 1-100 mg per film.

16. The oral dispersible film as claimed in claim 1, wherein the stabilizers employed are selected from maltodextrin, mannitol, glucose, lactose, dextrose, lactitol, erythrol or xylitol.

17. The oral dispersible film as claimed in claim 16, wherein the stabilizer is maltodextrin.

18. The oral dispersible film as claimed in claim 1, wherein the stabilizer is present at a concentration of 0.5-50 mg per film.

19. The oral dispersible film as claimed in claim 1, wherein the flavouring agents are selected from cherry, peppermint, mango, ginger, clove, spearmint or coffee with mint.

20. The oral dispersible film as claimed in claim 1, further comprises of preservatives, sweetening agents, and plasticizers.

21. The oral dispersible film as claimed in claim 20, wherein the preservatives are selected from sorbic acid/potassium sorbate, methyl-4-hydroxybenzoate and propyl-4-hydroxybenzoate.

22. The oral dispersible film as claimed in claim 21, wherein the preservative is Sodium benzoate.

23. The oral dispersible film as claimed in claim 20, wherein the preservative is present at a concentration of 1-50 mg per film.

24. The oral dispersible film as claimed in claim 20, wherein the sweetening agents are selected from the group comprising of mannitol, glucose, lactose, dextrose, lacitol, erythrol, xylitol, aspartame, neotame, sodium saccharin, or sucralose.

25. The oral dispersible film as claimed in claim 24, wherein the sweetening agent is sucralose.

26. The oral dispersible film as claimed in claim 20, wherein the sweetening agent is present at a concentration of 1-50 mg per film.

27. The oral dispersible film as claimed in claim 20, wherein the plasticiser is glycerine.

28. The oral dispersible film as claimed in claim 20, wherein the plasticiser is present at a concentration of 1-100 mg per film.

29. The oral dispersible film as claimed in claim 1, wherein water is added as a bulk medium.

30. The oral dispersible film as claimed in claim 1, wherein the thickness of the film is 0.6-0.8 mm.

31. The oral dispersible film as claimed in claim 1, wherein disintegration time of the film is between 30-60 seconds.

32. An oral dispersible film of Mitragynine, comprising:
i. Purified Mitragynine oxalate salt: 5-200 mg/film;
ii. Hydroxypropylmethyl cellulose (HPMC) and pullulan: 10-100 mg/film;
iii. Mannitol: 1-100 mg/film;
iv. Maltodextrin: 0.5-50 mg/film;
v. Flavouring agent selected from cherry, peppermint, mango, ginger, clove, spearmint, coffee with mint: 1-10 mg/film;
vi. Sodium benzoate: 1-50 mg/film;
vii. Sucralose: 1-50 mg/film;
viii. Glycerine: 1-100 mg/film; and
ix. Water: q.s.

33. The oral dispersible film as claimed in claim 32, wherein:
Thickness of film is 0.6-0.8 mm
Endurance is 3-4 kp
pH value: 2.0 to 3.2
Disintegration time is 50-60 sec.

34. The oral dispersible film as claimed in claim 32, wherein purified Mitragynine is obtained from Mitragyna speciosa.

35. A process for preparation of oral dispersible thin film of purified Mitragynine, comprising the following steps of:
a. mixing disintegrant, stabilizer, preservative, sweetening agent and plasticizer in 50% water, until a clear solution is formed;
b. adding the film forming polymer to the clear solution of step (a) followed by mixing to obtain a homogeneous mixture;
c. checking for lumps or insoluble material followed by mixing to obtain a clear solution;
d. uniformly mixing purified Mitragynine in 50% water, until a uniform dispersion is formed;
e. mixing clear solution of step (c) and uniform dispersion of Mitragynine of step (d) to obtain a uniform viscous solution;
f. adding flavouring agent to the uniform viscous solution of step (e) followed by mixing; and
g. coating and drying the viscous solution of step (f) to form thin oral dispersible film followed by packaging.

36. The process as claimed in claim 35, wherein the mixing is carried out with overhead stirrer.

37. The process as claimed in claim 35, wherein drying process is selected from spray drying, vacuum drying, freeze drying, tray drying, rotatory drying, drum drying, fluidized bed drying.

38. The process as claimed in claim 35, wherein the purified Mitragynine is Mitragynine free base or Mitragynine salt.

39. The process as claimed in claim 38, wherein the Mitragynine free base is in crystalline or amorphous form.

40. The process as claimed in claim 39, wherein the Mitragynine free base is in amorphous form.

41. The process as claimed in claim 38, wherein the purity of Mitragynine free base is 10-99% by HPLC.

42. The process as claimed in claim 38, wherein the purified Mitragynine salt is selected from purified Mitragynine hydrochloride salt, purified Mitragynine hydrobromide salt, purified Mitragynine perchlorate salt, purified Mitragynine sulphate salt, purified Mitragynine oxalate salt, purified Mitragynine tartrate salt, purified Mitragynine citrate salt, purified Mitragynine formate salt, purified Mitragynine trifluoro acetate salt, or purified Mitragynine trichloro acetate salt.

43. The process as claimed in claim 42, wherein the purified Mitragynine salt is purified Mitragynine oxalate salt.

44. The process as claimed in claim 38, wherein the purity of Mitragynine salt is 10-99% by HPLC.

45. The process as claimed in claim 35, wherein purified Mitragynine is obtained from Mitragyna speciosa.

46. The oral dispersible film as claimed in claim 1, wherein purified Mitragynine is obtained from Mitragyna speciosa.
